(19) United States
(12) Patent Application Publication
(10) Pub. No.: US 2012/0221064 A1
(43) Pub. Date: Aug. 30, 2012

(54) PROTECTIVE BARRIERS FOR MEDICAL DEVICES

(75) Inventor: Scott Wooldridge, Carmel, IN (US)

(73) Assignee: Roche Diagnostics Operations, Inc., Indianapolis, IN (US)

(21) Appl. No.: 13/036,352

(22) Filed: Feb. 28, 2011

(51) Int. Cl. A61N 1/00

(52) U.S. Cl. 607/2

(57) ABSTRACT

Exemplary protective barriers may be provided that house at least a portion of a medical device to prevent contamination of the medical device when it is used for treatment or testing. The protective barriers are removable from the medical device after use so that disinfection of the medical device is not required.

### TECHNICAL FIELD

The technical field relates to protective barriers to prevent contamination, and more particularly to such protective barriers for medical devices.

### BACKGROUND

Medical devices, particularly medical testing devices, are typically hand-held and used in a hospital, clinic, physician's office, ambulance, or other medical care location so that a physician, nurse, technician or other attendant can test biological samples or vital conditions obtained from a patient to determine an indication of a medical condition. It is desirable that the medical device be able to be re-used for other patients. Therefore, the medical device must be decontaminated and/or disinfected after each use to reduce the risk of spreading infection or disease between patients, and between the patients and the attendants.

Disinfectants and other cleaners can be used to de-contaminate a medical device after its use. However, certain medical devices include electronic or other sensitive components, push buttons, irregular or non-smooth surface features, and other features that make decontamination and disinfection difficult. In addition, disinfectants and other cleaners can degrade the components of the medical device, increasing the frequency in which repairs or replacements are needed, reducing product life, and increasing warranty costs to the manufacturer. Designing the electronic components and housing of the medical devices to withstand disinfection can increase the cost of the device. In addition, as new disinfectant products and cleaning protocols are identified, product maintenance costs are increased since the medical device must continually be tested and validated to determine and verify claims regarding the medical device's ability to be disinfected.

The use of disinfectants to decontaminate medical devices between uses can raise other problems. For example, disinfectants can be costly, require special training in their use, and special handling for storage and disposal. The use of disinfectants can also be time consuming and require a special location in which the decontamination is to be completed. The reliability of the disinfectant process can also vary based on the ability of the operator performing the decontamination and the process that is used. In addition, the owner of the meter must monitor the impact of repeated disinfection on the performance of the medical device, and deal with the manufacturer to obtain a repair or replacement medical device if performance has been unsuitably degraded.

Therefore, it is desirable to avoid or minimize the use of disinfectants in removing contamination from medical devices while preventing contamination during use of the medical device so it can be re-used at a later time.

### SUMMARY

Protective barriers are provided for medical devices. The protective barrier includes a casing that houses at least a portion of the medical device to prevent or reduce the possibility of contamination of the medical device by the user and/or patient during use. As used herein, the “user” can be a physician, nurse, attendant, caregiver, or person being treated with the medical device. The casing includes an outer surface, an inner surface, and at least one opening through which the medical device may be positioned. The casing may be designed to entirely enclose the medical device, or to partially enclose the medical device. When partially enclosed, it is contemplated that the casing provides a barrier on those portions of the medical device that are handled by the user or that are most likely to be exposed to the patient. In either situation, the casing is effective to prevent contamination of the medical device from bodily fluids, bacteria, viruses and other substances. Although not necessarily limited to such, the casings are useful for handheld medical devices having electronic components or other features that are degraded by the use of disinfectants and other cleaners applied directly to the medical device for disinfection and/or sterilization. The casing allows the use of disinfectant, cleaners and/or sterilizers to be avoided which may increase the useful life of the medical device, reduce product development and testing costs, and provide a more reliable decontamination regimen.

It is contemplated that the medical device is positioned in the casing before its use. For purposes of this disclosure, a “use” may include multiple uses of the device by a single test subject, or each singular use of the device among different individual test subjects. After each use, the casing is readily disposed of so that it cannot be re-used. According to one aspect, the casing must be destroyed in order to remove the medical device, preventing re-use of the casing in subsequent uses of the medical device. In one refinement of this aspect, the casing includes perforations between the opening and an end of the casing opposite the opening. The casing is severed or separated along the perforations, such as by manually tearing or cutting, to remove the medical device.

According to another aspect, the casing includes a flap adjacent one side of the opening that is folded or pulled over the opening and secured to the outer surface of the casing with an adhesive. The outer surface includes a plurality of demarcations indicating locations at which the flap may be secured to provide a custom-fit casing for a particularly sized medical device, allowing the same casing configuration to be used with different sizes of medical devices.

According to another aspect, an adhesive is provided generally about the circumference of the opening for securing the opening directly to the medical device, such that the medical device is only partially enveloped by the casing about those portions of the medical device which are desirably kept protected by the casing.

According to another aspect, the casing includes an end opposite the opening that is tapered along opposite sides of the casing so the medical device is properly located and registered in the casing. This can be useful when the casing includes a passage or other feature that is to be aligned with a portion of the medical device to facilitate use of the medical device during the procedure. In one refinement of this aspect, the tapered sides of the medical device extend to opposite corners that are crimped to provide handling features which may be handled by the user to facilitate destructive removal of the medical device from the casing.

These and other aspects, refinements, features, advantages, objects, and purposes of the protective barriers are discussed further below with reference to the drawings included herewith.

### BRIEF DESCRIPTION OF THE DRAWINGS

FIG. 1 is a proximal plan view of a first exemplary protective barrier for protecting a medical device from contamination during use.

FIG. 2 is a section view through line 2-2 of FIG. 1.

FIGS. 3A and 3B are sectional views through alternate embodiment perforations of the protective barrier of FIG. 1.

FIG. 4 is a bottom end elevation view of the protective barrier of FIG. 1.

FIG. 5 is a side elevation view of the protective barrier of FIG. 1.

FIG. 6A is a perspective view showing the protective barrier of FIG. 1 and a medical device positioned in the protective barrier.

FIG. 6B is a perspective view showing a modified protective barrier of FIG. 1 where the flap is secured to the distal side of the barrier with the medical device positioned in the modified protective barrier.

FIG. 7 is a perspective view showing the protective barrier of FIG. 1 destructively separated along its perforation to form an opening for removal of the medical device.

FIG. 8 is a perspective view showing the another embodiment of the protective barrier of FIG. 1 with its flap destructively detached from the outer surface of the protective barrier to form an opening for removal of the medical device.

FIG. 9 is a perspective view of an another exemplary protective barrier.

FIG. 10 is a perspective view of an another exemplary protective barrier and a medical device positioned in the protective barrier.

### DETAILED DESCRIPTION OF EXEMPLARY EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

A number of exemplary embodiments of the protective barrier comprise a casing configured to receive a medical device to prevent contamination of the medical device when the medical device is used for treatment. The protective barriers can be provided for protection with a variety of medical devices such as, for example, medical measurement devices such as blood-glucose meters, electronic blood pressure meters, electronic blood gas meters, electronic pulse/heart rate monitor, or multiple function meters which provide combinations of these and other functions. The protective barriers can also be provided for protection of medical devices with electrical components and movable parts, such as push buttons, key pads, scroll bars, knobs, and other features. Protection of the medical devices eliminates or reduces the need to apply disinfectants or other cleaners to the medical device after each use to eliminate biological and other contaminants that may have come in contact with the medical device during use. The protective barriers can be disposable to prevent their re-use in a subsequent procedure with the medical device. However, to ensure re-use is prevented, the protective barriers typically comprise one or more features that require destruction of the barrier as a result of removing the medical device from the barrier.

Exemplary protective barriers employ casings that are at least translucent if not transparent, at least in particular locations, so the user can visualize input components, display components, or other features of the medical device through the casing to enable complete use of the device. The casings may also be any one or combination of flexible, elastic, collapsible, foldable, arid pliable so that the shape of the casing readily receives, conforms to or adapts to the outer shape or profile of the medical device positioned in the casing. Suitable materials for the protective barriers include plastics and polymers such as polyethylene, polypropylene, polyester; elastomers; impermeable fabrics; foils; and/or latex-free materials. However, the protective barriers described herein are not limited to such materials.

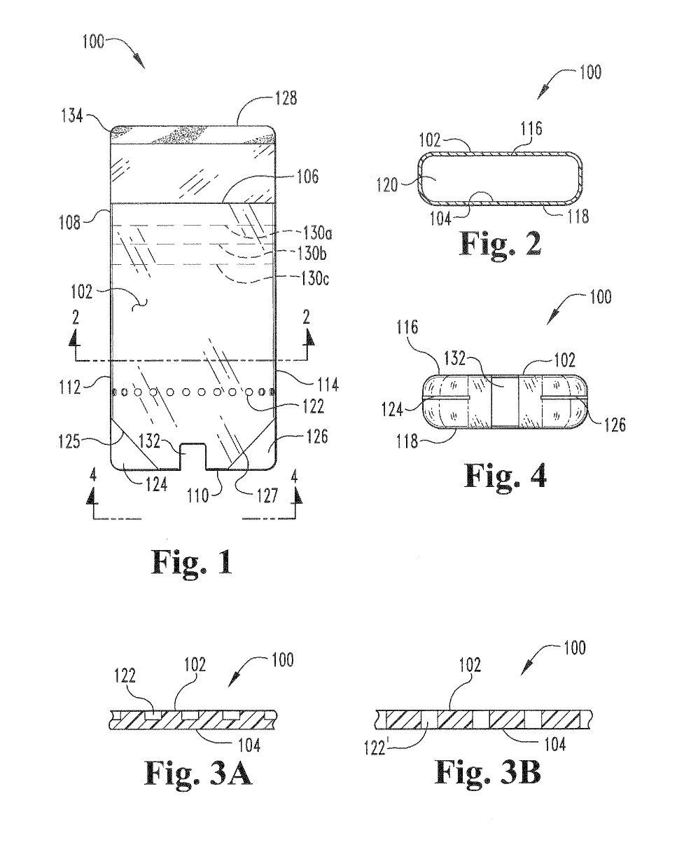

FIGS. 1-5 illustrate an exemplary protective barrier in the form of casing 100 for a hand-held medical device. Casing 100 includes outer surface 102, an inner surface 104 which defines an inner boundary of the casing 100 around space 120, and at least one opening 106 to provide access to space 120. Opening 106 is located at a first end 108 of casing 100. Casing 100 includes a second end 110 opposite first end 108, opposite lateral sides 112, 114 that extend between second end 110 and first end 108, and opposite proximal and distal sides 116, 118 that are defined between first and second ends 108, 110 and lateral sides 112, 114. Opening 106 is sized to allow a medical device to be positioned therethrough, and inner surface 104 is configured to receive at least a portion of a hand-held medical device through opening 106. Other embodiments contemplate that the opening 106 could be provided through second end 110, or through one of lateral sides 112, 114, or through one of the proximal or distal sides 116, 118.

In the embodiment of FIG. 1, casing 100 also includes perforations 122 at least across proximal side 116, although embodiments where perforations also or alternatively extend across distal side 118 and lateral sides 112, 114 are also contemplated. As shown in FIG. 3A, perforations 122 may not extend completely through outer and inner surfaces 102, 104 to maintain a complete protective barrier around the medical device while allowing separation of casing 100 along perforation 122, as discussed further below. Alternatively, as shown in FIG. 3B, at least some or all of perforations 122′ may extend completely though outer and inner surfaces 102, 104 to facilitate separation of casing 100 along perforations 122′, as discussed further below. In general, perforations are one possible feature of the casing that may be provided to facilitate destructively opening the casing for removal of a medical device provided therein, as discussed further below.

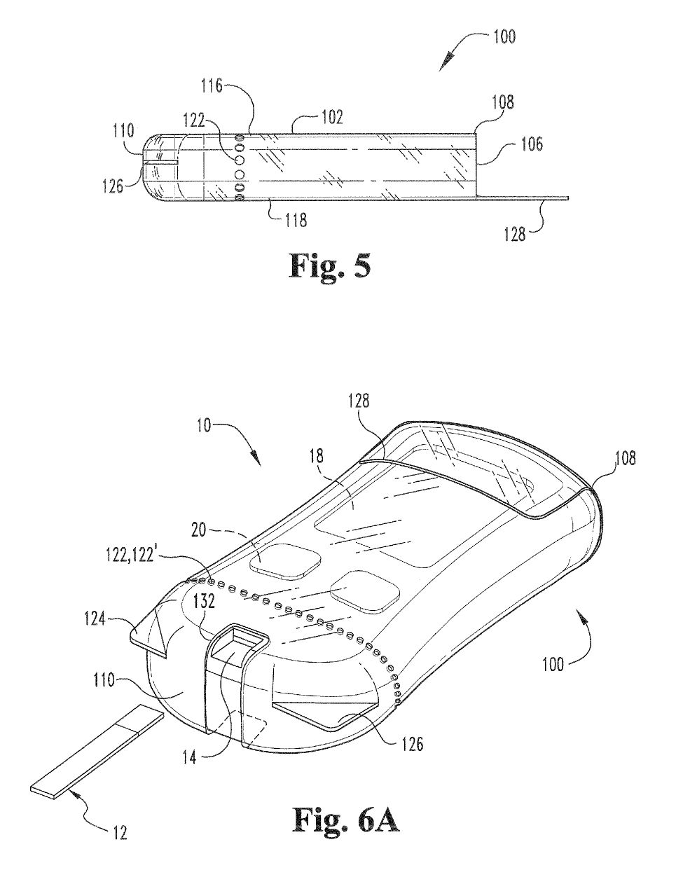

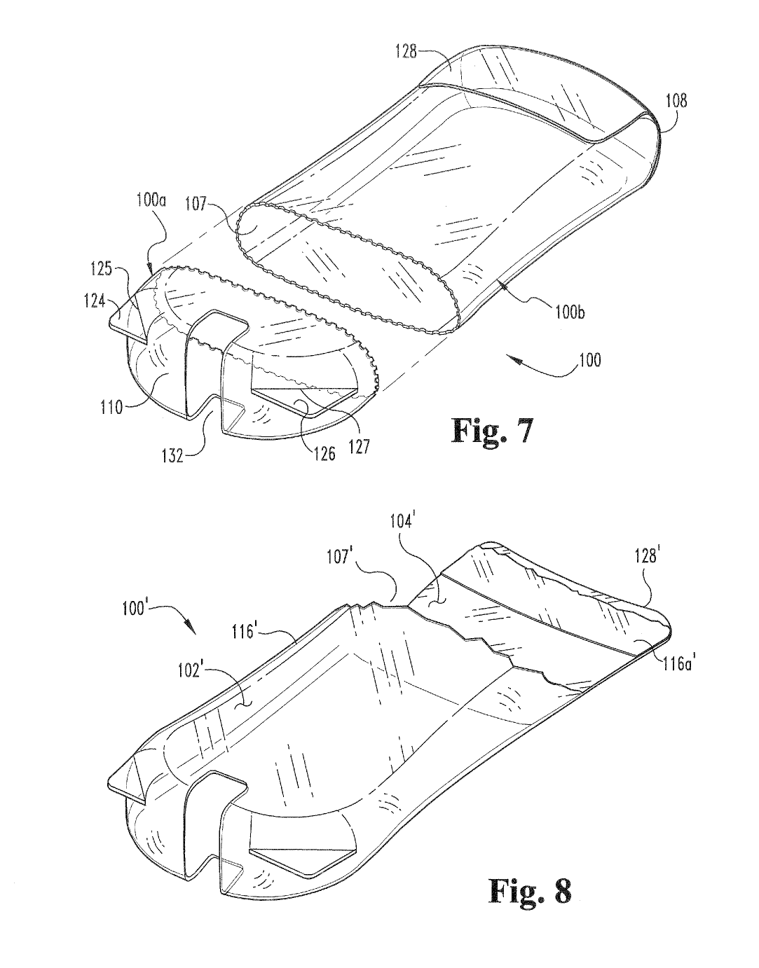

In one embodiment, the space 120 at second end 110 is generally tapered, for purposes set forth below. As further shown in FIGS. 4 and 5, second end 110 includes opposite corners 124, 126 formed by the taper of opposite lateral sides 112, 114 adjacent to second end 110. Corners 124, 126 can be formed by, for example, crimping, adhering, fusing, melting, or securing a portion of the inner surface 104 of the proximal and distal sides 116, 118 together at least along taper lines 125, 127 to form a flattened profile. Corners 124, 126 may be formed in other embodiments by securing inner surfaces 104 of proximal and distal sides 116, 118 only along their respective taper lines 125, 127, or by securing the inner surfaces 104 of proximal and distal sides 116, 118 along most or all of the inner surface area of the respective corners defined respectively by lines 125, 127, end 110 and edges 112, 114. In either arrangement, corners 124, 126 provide a handling feature in which the user may grasp casing 100 and destructively separate it along perforations 122, 122′. Referring to FIG. 7, this separation allows removal of the medical device from casing 100 while also destroying casing 100 so that is no longer suitable for use as a harrier. It is contemplated that removal of the medical device is facilitated when the secondary opening 107 created generally at the perforation is sufficiently large to allow the medical device to pass through the secondary opening 107. As used herein, the casing is destroyed when at least one of the ends or the sides is of the casing is no longer able to provide an appropriate protective barrier when the medical device is positioned therein. The user therefore is required to select another casing 100 to use the medical device in a subsequent procedure.

The tapered portions of lateral sides 112, 114 extending from second end 110 also guide insertion of the medical device to the proper location in casing 100. For example, the medical device may include a port or other receiving feature that requires insertion of an element into the medical device. The medical device may alternatively or additionally require projection of an element therefrom through casing 100. Thus, in certain embodiments, casing 100 includes a passage 132 opening through outer and inner surfaces 102, 104 at second end 110 that can be aligned with the respective receiving or projecting portion of the medical device. A passage 132 can be formed, for example, by punch cutting, die cutting, laser cutting, or any other appropriate technique for removing a portion of the distal and proximal sides 116, 118 at second end 110 to define the passage in the desired location for alignment with a medical device intended for use with the casing.

The tapered portions of lateral sides 112, 114 can be configured so that the medical device and its respective receiving or projecting portion are aligned with passage 132 when the medical device is properly positioned in casing 100. Taper lines 125, 127 may thus be selectively located in order to accommodate a variety of differently sized medical devices. Casing 100 may include an additional cover or flap (not shown) that is removably secured to casing 100 to allow the user to selectively open passage 132. Passage 132 may also be closed by such a cover or flap when access to the medical device is not needed or when access has been completed.

First end 108 of casing 100 may also include a flap 128 that can be folded, pulled or flexed over opening 106 at first end 108 to secure the medical device in space 120, such that the medical device is held in place adjacent to second end 110, for example abutting against the tapered portions of the lateral sides 112, 114 for proper location of the medical device within the space 120. At least one of flap 128 and the outer surface 102 includes an adhesive 134 that secures flap 128 to outer surface 102. In one embodiment, an adhesive backing strip (not shown) may be provided in connection with the adhesive 134 to prevent adhesion until desired by a user who removes the backing strip just prior to pulling the flap 128 over the opening 106. In the illustrated embodiment, adhesive 134 is shown on flap 128, although placement of adhesive on outer surface 102 in addition to or instead of on flap 128 is also contemplated. The adhesive 134 can be of sufficient strength so that flap 128 cannot be opened without destroying casing 100 to prevent its re-use. Alternatively, adhesive 134 can allow removal and reattachment of flap 128 to outer surface 102 to allow the user to remove or re-position the medical device in casing 100.

Outer surface 102 may also include a plurality of demarcations 130a, 130b, 130c that provide locations for the user to secure flap 128 to outer surface 102 and form a casing 100 of desired length. For example, demarcations 130a, 130b, 130c can correspond to lengths of various makes and/or models of medical device, allowing the user to easily secure flap 128 in a location along outer surface 102 that forms a casing 100 of optimal length from first end 108 to second end 110 for the particular make and/or model of the medical device positioned in casing 100.

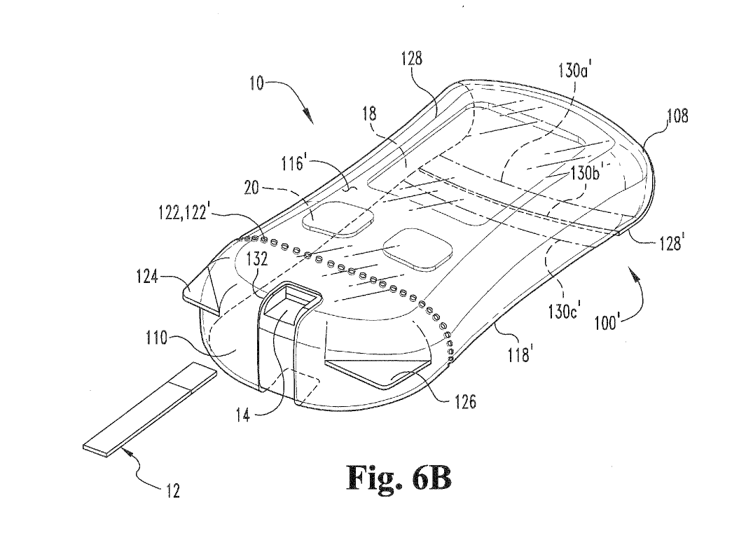

FIG. 6A shows a medical device 10 positioned in casing 100. In the illustrated embodiment, medical device 10 is a blood glucose meter that receives a test element 12 containing a blood sample. Medical device 10 includes a housing 16 that is entirely received in casing 100. However, as discussed further below, housing 16 and casing 100 may be configured so that only a portion of housing 16 is positioned in casing 100. Medical device 10 includes a test element receiving port 14 that is aligned with passage 132 of casing 100, allowing insertion of test element 12 through casing 100 and into receiving port 14. Medical device 10 also includes a display 18 and input means 20 that are visible through casing 100. The flexibility of casing 100 also allows the user to manipulate input means 20 through casing 100 so that the user can operate medical device 10 while it is in casing 100. Furthermore, display 18 is visible through casing 100 so that the user can visualize the output produced by medical device 10 during use.

Furthermore, although flap 128 is shown secured to proximal side 116 in FIG. 6A, FIG. 6B shows a modified casing 100′ that is substantially identical to casing 100 but includes a flap 128′ extending from proximal side 116′ so that flap 128′ can be secured to distal side 118′ of casing 100′. In addition, casing 100′ includes demarcations 130a′, 130b′, 130c′ across distal side 118′ to indicate locations where flap 128′ should be secured to casing 100′ for various makes and/or models of medical device 10. In the modified form of FIG. 6B, casing 100′ is arranged so that flap 128′ and demarcations 130a′-130c′ will not extend across display 18 and potentially obscure a portion of portions of display 18 from the user.

When use of the medical device 10 is complete, the user can grasp one or both of corners 124, 126 and separate casing 100 along perforations 122, 122′ to create second opening 107 large enough to remove medical device 10 from casing 100, such as shown in FIG. 7. In FIG. 7, opening 107 is created by completely detaching first casing portion 100a from second casing portion 100b. It is also contemplated that opening 107 can be created by only partially detaching casing portion 100a from easing portion 100b sufficiently to create opening 107 large enough to remove medical device 10. Alternatively or additionally, adhesive 134 may be configured to allow flap 128 to be opened to remove medical device 10 without destroying the casing 100.

In the illustrated embodiment, perforations 122, 122′ are shown located closer to second end 110 than first end 108 to facilitate removal of medical device 10 from casing 100 when casing 100 is separated along perforations 122, 122′. By providing perforations 122, 122′ closer to second end 110, the second end of medical device 10 can be pushed through the opening 107 that is created so that medical device 10 can be removed from casing 100. Other embodiments contemplate perforations 122, 122′ can be provided at the middle of or closer to first end 108. In still other embodiments, perforations 122, 122′ are provided at multiple locations across and/or along casing 100.

As shown in FIG. 8, another embodiment casing 100′ is shown that is similar to casing 100, but casing 100′ lacks perforations, although providing perforations is not precluded. Flap 128′ is shown detached from outer surface 102′ to create an opening 107′ for removal of the medical device. Casing 100′ is destructively opened since the adhesive on flap 128′ remains attached to a portion 116a′ of proximal side 116′ of casing 100′ after flap 128′ is opened to create opening 107′. Casing 100′ is thus torn or severed when flap 128′ is opened, preventing re-use of casing 100′.

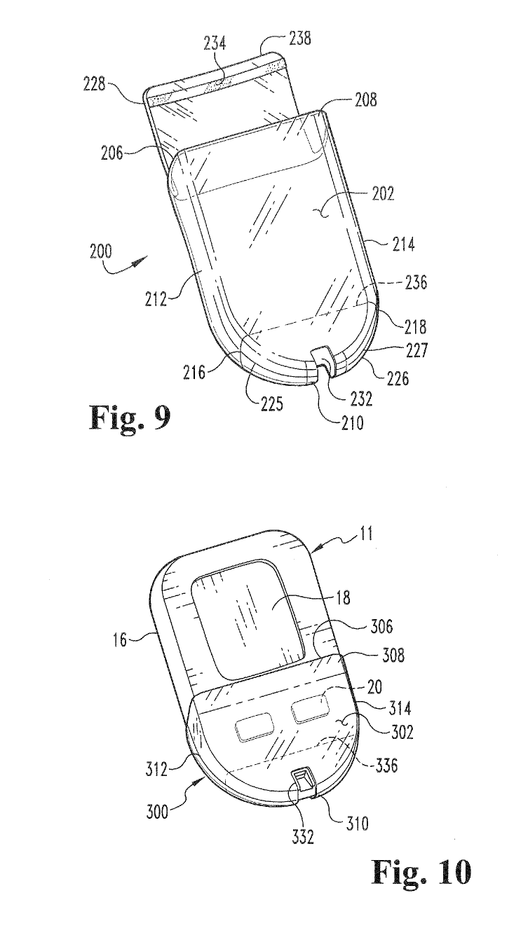

FIG. 9 shows another embodiment of casing 200 for a handheld medical device. Casing 200 includes an outer surface 202 and an opposite inner surface which defines an inner boundary of the casing 200. The inner boundary of casing 200 extends around a space that receives medical device 10, such as discussed above with respect to casing 100. Casing 200 includes at least one opening 206 to provide access to the inner boundary. In the illustrated embodiment, opening 206 is located at a first end 208 of casing 200. Casing 200 includes a second end 210 that is opposite first end 208, and opposite lateral sides 212, 214 extending between ends 208, 210. Other embodiments contemplate that the opening 206 could be provided through second end 210, or through one of lateral sides 212, 214, or through one of the proximal or distal sides of casing 200.

Lateral sides 212, 214 each include a respective one of opposite tapered portions 216, 218 extending from second end 210. The tapered portions 216, 218 of lateral sides 212, 214 extending from second end 210 also guide insertion of the medical device to the proper location in casing 200. For example, the medical device may include a port or other receiving feature that requires insertion of an element into the medical device through passage 232 of casing 200. The medical device may alternatively or additionally require projection of an element therefrom through passage 232 of casing 200. The tapered portions 216, 218 can be configured so that the medical device and its respective receiving or projecting portion are aligned with passage 232 when the medical device is properly positioned in easing 200. In addition, casing 200 may include an adhesive 236 on its inner surface adjacent to second end 210 and around passage 232. Adhesive 236 secures casing 200 to medical device 10 and maintains passage 232 in registry with port 14 of medical device 10 during use of medical device 10.

First end 208 of casing 200 may also include a flap 228 that can be folded, pulled or flexed over opening 206 at first end 208 to secure the medical device in casing 200. At least one of flap 228 and the outer surface 202 includes an adhesive 234 that secures flap 228 to outer surface 202. In the illustrated embodiment, adhesive 234 is shown on flap 228. The adhesive 234 can be of sufficient strength so that flap 228 cannot be opened without destroying casing 200 to prevent its re-use. Alternatively, adhesive 234 can allow removal and reattachment of flap 228 to allow the user to remove or re-position the medical device in casing 200. In addition, flap 228 can be provided with a tab portion 238 projecting from adhesive 234 that does not adhere to outer surface 202 to provide the user a location in which to grip flap 228 and open flap 228 when it is desired to remove or reposition medical device 10.

The tapered portions of the casing 200 of FIG. 9 are similar to those of casing 100 in the embodiments of FIGS. 1 and 6 but without the handling features provided by the corners 124 and 126. Such tapered portions of casing 200 may, for example, be formed in a similar fashion to defining the corners 124 and 126, followed by cutting or severing those corners generally at the taper lines 225, 227. In various embodiments, taper lines 125, 127 and 225, 227 may be straight (such as shown in FIG. 1 for lines 125, 127) or slightly curved (as shown in FIG. 9 for lines 225, 227).

FIG. 10 shows another embodiment casing 300 for a hand-held medical device. Casing 300 includes outer surface 302 and opposite inner surface which defines an inner boundary of the casing 300 around a space that receives only a portion of the medical device 11 that requires protection from exposure to test subjects, bodily fluids or other contaminating substances. Casing 300 includes at least one opening 306 to provide access to the inner boundary. In the illustrated embodiment, opening 306 is located at a first end 308 of casing 300. Casing 300 includes a second end 310 opposite first end 308, and opposite lateral sides 312, 314. Other embodiments contemplate that the opening 306 could be provided through second end 310, or through one of lateral sides 312, 314, or through one of the proximal or distal sides of casing 300.

Lateral sides 312, 314 include lengths that extend along only a portion of the medical device 11 from second end 310. The upper portion of medical device 11 that will not come into contact with the patient or fluids or other substances to be tested or sampled extends from casing 300 and is exposed. Casing 300 may include a passage 332 or other receiving feature that allows insertion of an element into the medical device. The medical device may alternatively or additionally require projection of an element therefrom through passage 332 of casing 300. In addition, casing 300 may include an adhesive 336 on its inner surface adjacent to second end 310 and around passage 332. Adhesive 336 secures casing 300 to medical device 11 and maintains passage 332 in registry with port 14 of medical device 11 during use of the medical device.

In any of the embodiments discussed above, the adhesives may be provided with a temporary cover to prevent inadvertent adherence of the flap to the medical device or to the cover until the medical device is located in the desired position in the casing. The temporary cover can be removed when it is desired to seal with the adhesive. Any of the casings discussed herein can be provided in a kit or set comprising a plurality of casings that are individually selected and removed from a container or roll for use with the medical device. The plurality of casings can be maintained in an unconnected manner, or connected to one another in an end-to-end manner with a perforated connection between adjacent casings.

In accordance with the foregoing description of embodiments of a casing, methods have also been conceived relating to protecting a medical device from contaminating substances, and relating to hygienic removal of a casing from a medical device in order to prevent its re-use.

In order to protect a medical device from contaminating substances, a casing is provided having some or all of the features of the embodiments of casings described herein. A medical device is provided and inserted through an opening in a first end of the casing so that an end of the medical device abuts the second end of the casing. If a passage is provided in the casing for insertion of a test element or other adjuvant to the medical device, then the interacting portion of the medical device is located so that it is aligned with the passage in the casing. In one embodiment, location is accomplished by means of tapered portions provided in the second end of the casing. The opening is then sealed either by securing a flap of the casing over the opening, or by securing the inner surface of the casing directly to the medical device, such securing by means of, for example, an adhesive provided on a portion of the flap or the inner surface of the opening. The casing comprises a transparent or translucent material so that the medical device provided in the casing may be used without any visual hindrance. The medical device is then used by a test subject or health care provider.

It has been generally described above a manner for removing the casing from the medical device, generally by detaching the flap at the opening or by separating the casing along perforations in the outer surface of the casing in order to form a second opening. However, it is also desirable and important to maintain a hygienic status of the meter during such removal. That is, if the second opening is formed, for example, and the exposed medical device is immediately contacted by a contaminated surface such as the outside of a medical glove or by an unprotected hand that has handled the outside surface of the casing, then the purpose of the casing has been rendered moot and disinfection of the device will still be required. Thus, in one embodiment, a method for a person to remove the casing comprises the steps of covering the person's hands with medical gloves or other contamination-resistant hand coverings; with a first hand, holding or otherwise grasping the casing-enclosed medical device; using the second hand, exposing a portion of the medical device by one of destructively detaching the flap covering the opening and destructively separating the casing such as at a perforation in the outer surface to form a second opening large enough for the medical device to be removed therethrough; uncovering the first hand holding the enclosed medical device by inside-out removal of the hand-covering over the grasped portion of the enclosed medical device; using the second hand, grasping the enclosed medical device about the inside-out hand-covering removed from the first hand; using the uncovered first hand, grasping the exposed portion of the medical device and setting the medical device aside; and using the uncovered first hand, uncovering the second hand by inside-out removal of the hand covering over the inside-out covering of the first hand; and disposing of the hand coverings and casing.

It is noted that terms like “preferably”, “commonly”, and “typically” if utilized herein are not intended to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, patient, etc.) using the medical device. For example, the portion of a medical device facing the user would be proximal and the portion of the medical device facing away from the user would be distal.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modifications and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. An apparatus comprising:
a casing having an inner surface defining a space configured to receive a hand-held medical device, an outer surface, and a sealable opening providing access to the inner surface so that the medical device is positionable into the casing through the opening; and
the casing including a plurality of perforations extending into the outer surface at a location spaced from the opening;
wherein the casing is destructively separable along the perforations to remove the medical device from the casing after the medical device is used.

2. The apparatus according to claim 1 wherein the casing includes an end opposite the opening and the perforations are located between the opening and the end of the casing.

3. The apparatus according to claim 2 wherein the end opposite the opening is substantially closed to prevent the medical device from passing through the end opposite the opening.

4. The apparatus according to claim 3 wherein opposite lateral sides of the casing are tapered to the end opposite the opening to align the medical device with a passage extending through the end, the passage for receiving a test element insertable into the medical device.

5. The apparatus according to claim 1 wherein the casing is tapered along opposite lateral sides of the casing to form opposite corners at the end of the casing opposite the opening, the corners extending from respective ones of the tapered lateral sides, wherein the corners of the casing are formed having a generally flattened profile along the respective tapered lateral sides to provide a handling feature for applying a force to separate the casing along the perforations.

6. The apparatus according to claim 5 wherein sealable opening comprises a flap adjacent at least one side of the opening and positionable over the opening and securable to the outer surface so that the medical device is substantially enclosed in the casing.

7. The apparatus according to claim 6 wherein the casing includes an adhesive on at least one of the flap and the outer surface to secure the flap to the outer surface.

8. The apparatus according to claim 7 wherein the casing includes a plurality of target markings visible from the outer surface, the target markings providing selected locations along the casing in which to secure the flap to the outer surface to vary a length of the casing based on a length of the medical device positioned in the casing.

9. The apparatus according to claim 1 wherein the sealable opening comprises a flap adjacent at least one side of the opening and positionable over the opening and securable to the outer surface so that the medical device is substantially enclosed in the casing.

10. The apparatus according to claim 9 wherein the casing includes an adhesive on at least one of the flap and the outer surface to secure the flap to the outer surface.

11. The apparatus according to claim 10 wherein the casing includes a plurality of target markings visible from the outer surface, the target markings providing selected locations along the casing in which to secure the flap to the outer surface to vary a length of the casing based on a length of the medical device positioned in the casing.

12. The apparatus according to claim 1 wherein the casing is at least partially translucent so that at least a portion of the medical is visible through the casing.

13. The apparatus according to claim 1 wherein the casing completely encloses the medical device.

14. The apparatus according to claim 1, wherein the perforations extend partially into the casing.

15. The apparatus according to claim 1, wherein the perforations extend through the inner and outer surfaces of the casing.

16. The apparatus according to claim 1, wherein the perforations extend completely across the casing to opposite lateral sides of the casing.

17. An apparatus comprising:
a casing for a hand-held medical device, the casing having an inner surface, an outer surface, and an opening providing access to the inner surface so that the medical device is positionable into the casing through the opening; and
the casing including a second end opposite the opening and a flap adjacent to the opening;
wherein the casing includes a plurality of target markings visible from the outer surface, the target markings providing selected locations along the casing in which to secure the flap to the outer surface to vary a length of the casing based on a length of the medical device positioned in the casing.

18. The apparatus according to claim 17 wherein the casing includes a plurality of perforations extending into the outer surface at a location spaced from the opening and the second end, and the casing is separable along the perforations to remove the medical device from the casing after the medical device is used to destroy the casing to prevent its re-use with the medical device.

19. The apparatus according to claim 18 wherein the perforations extend through the inner and outer surfaces of the casing.

20. The apparatus according to claim 17 wherein the casing includes an adhesive on at least one of the flap and the outer surface to secure the flap to the outer surface.

21. The apparatus according to claim 17 wherein the second end of the casing is tapered along opposite lateral sides of the casing to align the medical device with a passage extending through the second end of the casing, the passage for receiving a test element insertable into the medical device.

22. The apparatus according to claim 17 wherein the casing is tapered along opposite lateral sides of the casing to form opposite corners extending from respective ones of the tapered lateral sides at the second end of the casing, wherein the opposite corners of the casing are crimped along the respective tapered lateral side to provide a handling feature for applying a force to separate the casing along the perforation.

23. An apparatus comprising:
a casing for a hand-held medical device, the casing having an inner surface, an outer surface, and an opening providing access to the inner surface so that the medical device is positionable into the casing through the opening; and
the casing including an end opposite the opening and opposite lateral sides extending between the opening and the end;
wherein the opposite lateral sides each include a tapered portion extending from the end opposite the opening toward the opening, the tapered portions aligning the medical device in the casing.

24. The apparatus according to claim 23 wherein the casing includes a plurality of perforations extending into the outer surface at a location between the opening and the end, and the casing is separable along the perforations to remove the medical device from the casing after the medical device is used to destroy the casing to prevent its re-use with the medical device.

25. The apparatus according to claim 24 wherein the casing includes opposite corners extending along the tapered portion of respective ones of the opposite lateral sides, and the opposite corners are crimped at least along the respective tapered portion to provide a handling feature for applying force to separate the casing along the perforation.

26. The apparatus according to claim 25 wherein the opposite corners are crimped by securing facing portions of the inner surface of the casing together.

27. The apparatus according to claim 23 wherein the casing includes a flap positionable over the opening so that the medical device is substantially enclosed in the casing and the casing includes an adhesive on at least one of the flap and the outer surface to secure the flap to the outer surface.
